(19) United States
(12) Patent Application Publication
(10) Pub. No.: US 2009/0124709 A1
(43) Pub. Date: May 14, 2009

(54) Analytical Methods for Validating Excipient Purity

(76) Inventors: Douglas L. Cole, Half Moon Bay, CA (US); Jing J. Zhang, Palo Alto, CA (US)

Correspondence Address: Goodwin Procter LLP;Attn: Patent Administrator, 135 Commonwealth Drive, Menlo Park, CA, 94025-1105, US

(21) Appl. No.: 12/207,378

(22) Filed: Sep. 9, 2008

Related U.S. Application Data
(60) Provisional application No. 60/971,196, filed on Sep. 10, 2007.

(51) Int. Cl. A61K 47/00

(52) U.S. Cl. 514/786; 514/788; 536/127; 73/61.55

(57) ABSTRACT

The invention concerns methods of validating the purity of n-Dodecyl beta-D-maltoside (DDM), a membrane permeation enhancer used in nasal and oral drug delivery methods, using HPLC and Mass Spectrometry techniques.

### CROSS-REFERENCE TO RELATED APPLICATIONS

This application is a U.S. Non-Provisional Application claiming priority under 35 U.S.C. § 119(e) to U.S. Provisional Application Ser. No. 60/971,196, filed on Sep. 10, 2007, which is incorporated by reference in its entirety.

### FIELD OF THE INVENTION

The invention concerns methods of validating the purity of n-Dodecyl beta-D-maltoside (DDM), a membrane permeation enhancer used in nasal and oral drug delivery methods, using HPLC and Mass Spectrometry techniques.

### BACKGROUND OF THE INVENTION

Candidate drugs possess a wide range of molecular size, shape, and chemical properties. Variations in the structure and chemistry of both the drug and the skin and mucous membranes contribute to the unpredictable nature of drug delivery. There is a recognized need to overcome the natural barrier properties of bodily membranes and skin in achieving drug bioavailability.

An additional hurdle that must be overcome in drug development is obtaining the purity level required for marketing approval.

### SUMMARY OF THE INVENTION

In light of the recognized need to overcome the barrier in achieving the required level of purity, the present invention relates to methods of analyzing the purity of permeation-enhancer DDM in a sample containing DDM. Specifically, the application provides methods of validating the purity of DDM using HPLC and Mass Spectrometry techniques.

In one aspect, the application provides a method for determining the purity of DDM in a DDM containing sample.

In another aspect, the application provides a method for determining the purity of DDM in a DDM containing sample by chromatography.

In another aspect, the application provides a method for determining the purity of DDM in a DDM containing sample by reverse phase HPLC.

In another aspect, the application provides a method for determining the purity of DDM in a DDM containing sample by HPLC-RI, HPLC-Corona CAD, HPLC-ELSD, GC, HPLC-MS, HPLC-MS-MS, or any combination thereof.

In another aspect, the application provides a method for determining the purity of DDM in a DDM containing sample by a method comprising the steps:
a) providing a reference standard containing DDM;
b) measuring the purity of DDM in the reference standard containing DDM by chromatography;
c) providing a sample containing DDM;
d) measuring the purity of DDM in the sample containing DDM by chromatography; and
e) comparing the difference between the purity of DDM in the reference standard containing DDM and the purity of DDM in the sample containing DDM.

In one aspect of the above method, the chromatography is HPLC-RI, HPLC-Corona CAD, HPLC-ELSD, GC, HPLC-MS, HPLC-MS-MS, or a combination thereof.

In one aspect of the above method, the chromatography is HPLC-RI.

In one aspect of the above method, the chromatography is HPLC-Corona CAD.

In one aspect of the above method, the chromatography is HPLC-ELSD.

In one aspect of the above method, the chromatography is GC.

In one aspect of the above method, the chromatography is HPLC-MS.

In one aspect of the above method, the chromatography is HPLC-MS-MS.

In one aspect, the application provides a method comprising the steps the steps:

a) providing a drug to be administered; and

b) adding DDM to said drug to be administered;

whereby the membrane permeability of said drug to be administered is enhanced.

In another aspect, the application provides a composition of matter for increasing the permeability of a body surface or membrane to at least one drug comprising said drug in combination with DDM, of purity as validated by any of the above methods, said DDM present in a permeation-enhancing amount sufficient to substantially increase the permeability of the body surface or membrane to at least one drug in order to deliver said drug to an individual at a therapeutically effective rate.

In one aspect of the above method, the DDM and at least one drug are dispersed within a pharmaceutically acceptable carrier.

In another aspect of the above method, the DDM is combined with a permeation-enhancing amount of one or more permeation enhancers selected from monoglycerides or mixtures of monoglycerides of fatty acids, lauramide diethanolamine, lower C₁₋₄ alcohols, alkyl laurates, acyl lactylates, dodecyl acetate, and C₁₀-C₂₀fatty acid esters.

In one aspect of the above method, the DDM is combined with a permeation-enhancing amount of one or more permeation enhancers selected from glycerol monolaurate, glycerol monooleate, glycerol monolinoleate, lauramide diethanolamine, ethanol, methyl laurate, caproyl lactylic acid, lauroyl lactylic acid, dodecyl acetate, and lauryl lactate.

In one aspect, the application provides a method comprising the steps:

a) providing a sample comprising DDM; and

b) using chromatography to separate the α-anomer and the β-anomer of DDM.

In one aspect of the above method, the chromatography is reverse phase HPLC.

In another aspect of the above method, the chromatography is HPLC-MS or HPLC-MS-MS.

In one aspect, the application provides a method comprising the steps:

a) providing a sample comprising DDM; and

b) using chromatography to detect partially deacetylated DDM.

In one aspect of the above method, the chromatography is reverse phase HPLC.

In another aspect of the above method, the chromatography is HPLC-MS or HPLC-MS-MS.

In one aspect, the application provides a method comprising the steps:

a) providing a sample comprising DDM; and

b) using chromatography to detect dodecanol.

In one aspect of the above method, the chromatography is reverse phase HPLC.

In another aspect of the above method, the chromatography is HPLC-MS or HPLC-RI.

In one aspect, the application provides a method comprising the steps:

a) providing a sample comprising DDM; and

b) using chromatography to detect 1-bromo or 1-chloro acylated maltose.

In one aspect of the above method, the chromatography is HPLC.

In another aspect of the above method, the HPLC is HPLC-MS or HPLC-MS-MS.

In one aspect, the application provides a method comprising the steps:

a) providing a sample comprising DDM; and

b) using chromatography to detect acyl maltose 1-ol.

In one aspect of the above method, the chromatography is HPLC.

In another aspect of the above method, the chromatography is HPLC-RI, HPLC-ELSD, HPLC-Corona CAD, GC, HPLC-MS or HPLC-MS-MS.

In one aspect, the application provides a method comprising the steps:

a) providing a sample comprising DDM; and

b) using chromatography to detect DDM homologues.

In one aspect of the above method, the chromatography is HPLC.

In another aspect of the above method, the chromatography is HPLC-RI, HPLC-ELSD, HPLC-Corona CAD, GC, HPLC-MS or HPLC-MS-MS.

In one aspect, the application provides a method comprising the steps administering to a patient a composition comprising a pharmaceutically effective amount of a drug for treating the condition and DDM, of purity as validated by any of the above methods, in a permeation enhancing-effective amount.

In one aspect, the application provides the use of DDM, of purity as validated by any of the above methods, as a permeation enhancing agent.

In another aspect, the application provides a method of treating a condition in a patient comprising administering to the patient a composition comprising a pharmaceutically effective amount of a drug for treating the condition and DDM, of purity as validated by any of the above methods, in an enhancing-effective amount.

### DETAILED DESCRIPTION

All publications, issued patents, and patent applications cited herein are hereby incorporated by reference.

As used in the present specification the following terms have the meanings indicated:

### DEFINITIONS

As used herein, the term “chromatography” refers to HPLC-RI, HPLC-ELSD, HPLC-Corona CAD, GC, HPLC-MS, HPLC-MS-MS, or any combination thereof.

As used herein, the term “drug” is intended to refer to a chemical entity, whether in the solid, liquid, or gaseous phase which is capable of providing a desired therapeutic effect when administered to a subject. The term “drug” should be read to include synthetic compounds, natural products and macromolecular entities such as peptides, polypeptides, polynucleotides, or lipids and also small entities such as neurotransmitters, ligands, hormones or elemental compounds. The term “drug” is meant to refer to that compound whether it is in a crude mixture or purified and isolated.

As used herein, the term “permeation enhancement” mean an increase in the permeability of a biological membrane (i.e. skin or mucosa) to a drug, so as to increase the rate at which the drug permeates through the membrane. “Permeation enhancer,” “enhancer,” “penetration enhancer,” or similar term means a material that achieves such permeation enhancement, and an “effective amount” of an enhancer means an amount effective to enhance penetration through the skin or mucosa of a selected agent to a selected degree.

As used herein, “reference standard” means a normalized value obtained from a standardized sample, and in the case of analyzing DDM containing samples, means the normalized maltose fragment ion determined by MS measured in a reference sample of known purity, which is compared in a parallel assay, by the same steps and conditions, to the tested sample containing DDM.

As used herein, the term “impurity” means an impurity of DDM, including without limitation, the n-Dodecyl maltoside α-anomer, partially deacylated DDM process-related impurities, free dodecanol or dodecanol process-related impurities, partially deacylated DDM process-related impurities, 1-Bromo or 1-chloro acylated maltose, acyl maltose-1-ol, DDM homologues, or any combination thereof.

As used herein, the term “substantially free” refers to the amount of one or more DDM impurities present in a sample containing DDM that would be deemed acceptable by a federal drug regulatory agency, such as the Food and Drug Administration.

As used herein, the term “comparable” means within limits deemed acceptable by any federal regulatory agency such as the Food and Drug Administration. Alternatively, or in the absence of such limits deemed acceptable by a regulatory agency to serve as guidelines, the term “comparable” means the same as the reference standard or within the same order of magnitude as the reference standard.

As used herein, the term “excipient” means pharmaceutically acceptable excipients, including carriers, stabilizers, and permeation enhancers that are non-toxic to the cell or mammal being exposed thereto at the dosages and concentrations employed. Examples of suitable excipients will be appreciated by those of skill in the art, such as those described Remington's Pharmaceutical Sciences 20^{th} edition, Gennaro, A. R. Ed. (2003), hereby incorporated by reference in its entirety.

A new drug, biological agent, therapeutic device or other potentially therapeutic substance or treatment must meet the approval of a government authority before it is marketed or commercially available to the public, In the United States, the Food and Drug Administration (FDA), has the authority to approve the use or sale of such products. The general procedure for gaining FDA approval involves developing a new drug and obtaining approval from the FDA (see U.S. Pat. No. 6,925,599, hereby incorporated by reference in its entirety). The relative purity of drugs and excipients is a measurement required by the FDA.

The present invention concerns methods of validating the purity of DDM using the techniques disclosed herein. In one embodiment, the method includes the steps of

a) providing a reference standard containing DDM;

b) measuring the purity of DDM in the reference standard containing DDM by chromatography;

c) providing a sample containing DDM;

d) measuring the purity of DDM in the sample containing DDM by chromatography; and

e) comparing the difference between the (i) purity of DDM in the reference standard containing DDM and the (ii) purity of DDM in the sample containing DDM; and

f) determining that the purity of DDM in the sample containing DDM is comparable to the purity of DDM in the reference standard. In some embodiments, the determining step includes determining that the DDM in the sample containing DDM is free of impurities or substantially free of impurities.

Measurement of the purity of DDM in a DDM containing sample may be performed by the techniques described herein. In some embodiments, measurements may be made using a software program executed by a suitable processor. Suitable software and processors are well known in the art and are commercially available. The program may be embodied in software stored on a tangible medium such as CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

Following the measurement of the purity of DDM in a DDM containing sample and the determination that a sample is free of impurities or substantially free of impurities, the measurement results or are typically recorded and communicated to technicians, clients, and/or government agencies, for example. In certain embodiments, computers will be used to communicate such information to interested parties, such as, technicians, clients, and/or government officials. In some embodiments, the assays will be performed or the assay results analyzed in a country or jurisdiction which differs from the country or jurisdiction to which the results are communicated.

In a preferred embodiment, a measurement based on the amount of impurities in a sample containing DDM herein is communicated to a technician, client, or government agency subject as soon as possible after the assay is completed and the determining step is generated. The results and/or related information may be communicated to the technician. Alternatively, the results may be communicated directly to a client by any means of communication, including writing, electronic forms of communication, such as email, or telephone. Communication may be facilitated by use of a computed, such as in case of email communications. In certain embodiments, the communication containing results of a measurement of impurities and/or conclusions drawn from, may be generated and delivered automatically to the subject using a combination of computer hardware and software which will be familiar to artisans skilled in telecommunications. One example of a system for communicating purity analysis is described in U.S. Pat. No. 7,197,405 (hereby incorporated by reference in its entirety); however, the present invention is not limited to methods which utilize this particular communications system. In certain embodiments of the methods of the invention, all or some of the method steps, including the assaying of samples, determining of impurity levels, and communicating of assay results or diagnoses, may be carried out in diverse (e.g., foreign) jurisdictions.

The present invention will now be described in connection with certain Examples, which are not intended to limit the scope of the invention. On the contrary, the present invention covers all alternatives, modifications, and equivalents as can be included within the scope of the claims. Thus, the following will illustrate the practice of the present invention, for the purposes of illustration of certain embodiments and is presented to provide what is believed to be a useful and readily understood description of its procedures and conceptual aspects.

### EXAMPLES

### Example 1

DDM Area-% Purity is determined by HPLC-RI and HPLC-Corona CAD for control and by HPLC-ELSD, GC and HPLC-MS-MS for verification of impurity profile and structures. Specifically, by detection of a fragment ion representative of the maltose moiety and MS selected ion monitoring, the Area-% Purity of DDM is determined.

### Example 2

n-Dodecyl maltoside a-anomer impurity level is detected by HPLC-RI, HPLC-ELSD, and HPLC-Corona CAD for control GC and HPLC-MS-MS for verification of impurity profile and structures. The α-anomer of DDM, a process-related impurity in finished product, is separated from the predominant β-anomer by reverse phase HPLC on a suitable column and detected by LC-MS in selected ion monitoring mode, then quantified in its ratio to the β-anomer.

### Example 3

Partially deacylated DDM process-related impurities are detected by HPLC-RI, HPLC-ELSD, HPLC-Corona CAD, GC and HPLC-MS-MS for verification of impurity profile and structure determination. These impurities in DDM are due to incomplete deacetylation following attachment of the dodecyl side chain ether. They are separated group-wise by RP-HPLC and quantified relative to DDM by selected ion monitoring LC-MS.

### Example 4

Free dodecanol process-related impurities are detected by HPLC-RI, HPLC-ELSD, and HPLC-Corona CAD for control, and GC or HPLC-MS-MS for impurity profile verification and structure determination. This process-related impurity arises from incompletely reacted dodecanol in final DDM, and is quantified by HPLC-MS or HPLC-RI.

### Example 5

1-Bromo or 1-chloro acylated maltose is detected by HPLC-MS or HPLC-MS-MS. This low-level impurity, potentially remaining after DDM workup, may be quantified by selected ion monitoring LC-MS.

### Example 6

Acyl maltose-1-ol is detected by HPLC-RI, HPLC-ELSD, HPLC-Corona CAD, GC or HPLC-MS-MS. This hydrolysis product of 1-halomaltose is quantified by HPLC separation and MS selected ion monitoring.

### Example 7

DDM homologs are detected by HPLC-RI, HPLC-ELSD, HPLC-Corona CAD, GC or HPLC-MS-MS. The C₁₂ sidechain of DDM is typically derived from petroleum-based raw materials that have been purified by distillation and thus may contain homolog impurities such as C₁₀, C₁₄, and the like. These may be quantified in finished DDM by HPLC-MS (MS).

### Summary of Examples

The purity of DDM may be determined by the methods shown in Table 1, for each of the analytical figures of merit listed in the left hand column.

TABLE I

| Analytical Figure of Merit | Analytical Method | Example of Technique |
|---|---|---|
| DDM Area-% Purity | Analysis by HPLC-RI and | By detection of a fragment ion |
| | HPLC-Corona CAD for control | representative of the maltose |
| | and by HPLC-ELSD, GC and | moiety and MS selected ion |
| | HPLC-MS-MS for verification | monitoring, the Area-% Purity |
| | of impurity profile and | of DDM is determined. This is |
| | structures | superior to previously reported |
| | | HLPC-RI methods that suffer |
| | | from low sensitivity for |
| | | impurity peaks. |
| n-Dodecyl maltoside α-anomer | HPLC-RI, HPLC-ELSD, and | The α-anomer of DDM, a |
| impurity level | HPLC-Corona CAD for control | process-related impurity in |
| | GC and HPLC-MS-MS for | finished product, may be |
| | verification of impurity profile | separated from the predominant |
| | and structures | β-anomer by reverse phase |
| | | HPLC on a suitable column |
| | | and detected by LC-MS in |
| | | selected ion monitoring mode, |
| | | then quantified in its ratio to |
| | | the β-anomer. |
| Partially deacylated DDM | HPLC-RI, HPLC-ELSD, | These impurities in DDM are |
| process-related impurities | HPLC-Corona CAD, GC and | due to incomplete deacetylation |
| | HPLC-MS-MS for verification | following attachment of the |
| | of impurity profile and | dodecyl side chain ether. They |
| | structure determination | are separated group-wise by |
| | | RP-HPLC and quantified |
| | | relative to DDM by selected |
| | | ion monitoring LC-MS. |
| Free dodecanol process-related | HPLC-RI, HPLC-ELSD, and | This process-related impurity |
| impurity | HPLC-Corona CAD for | arises from incompletely |
| | control, and GC or HPLC-MS- | reacted dodecanol in final |
| | MS for impurity profile | DDM, and may be quantified |
| | verification and structure | by HPLC-MS or HPLC-RI. |
| | determination | |
| 1-Bromo or 1-chloro acylated | HPLC-MS or HPLC-MS-MS | This low-level impurity, |
| maltose | | potentially remaining after |
| | | DDM workup, may be |
| | | quantified by selected ion |
| | | monitoring LC-MS. |
| Acyl maltose-1-ol | HPLC-RI, HPLC-ELSD, | This hydrolysis product of 1- |
| | HPLC-Corona CAD, GC or | halomaltose may be quantified |
| | HPLC-MS-MS | by HPLC separation and MS |
| | | selected ion monitoring. |
| DDM homologs | HPLC-RI, HPLC-ELSD, | The C₁₂ sidechain of DDM is |
| | HPLC-Corona CAD, GC or | typically derived from |
| | HPLC-MS-MS | petroleum-based raw materials |
| | | that have been purified by |
| | | distillation and thus may |
| | | contain homolog impurities |
| | | such as C₁₀, C₁₄, and the like. |
| | | These may be quantified in |
| | | finished DDM by HPLC- |
| | | MS(MS). |

It will be appreciated by those skilled in the art that changes could be made to the embodiments described above without departing from the broad inventive concept thereof. It is understood, therefore that this invention is not limited to the particular embodiments disclosed, but it is intended to cover modifications that are within the spirit and scope of the invention, as defined by the appended claims.

What is claimed:

1. A method comprising the steps:
a) providing a reference standard containing DDM;
b) measuring the purity of DDM in the reference standard containing DDM by chromatography;
c) providing a sample containing DDM;
d) measuring the purity of DDM in the sample containing DDM by chromatography;
e) comparing the difference between the purity of DDM in the reference standard containing DDM and the purity of DDM in the sample containing DDM; and
f) determining that the purity of DDM in the sample containing DDM is comparable to the purity of DDM in the reference standard.

2. The method of claim 1 wherein the chromatography is HPLC-RI, HPLC-Corona CAD, HPLC-ELSD, GC, HPLC-MS, HPLC-MS-MS, or a combination thereof.

3. The method of claim 2 wherein the chromatography is HPLC-RI.

4. The method of claim 2 wherein the chromatography is HPLC-Corona CAD.

5. The method of claim 2 wherein the chromatography is HPLC-ELSD.

6. The method of claim 2 wherein the chromatography is GC.

7. The method of claim 2 wherein the chromatography is HPLC-MS.

8. The method of claim 1 wherein the chromatography is HPLC-MS-MS.

9. A method comprising the steps:
a) providing a drug to be administered; and
b) adding DDM to said drug to be administered;
whereby the membrane permeability of said drug to be administered is enhanced.

10. A composition of matter for increasing the permeability of a body surface or membrane to at least one drug comprising said drug in combination with DDM, of purity as validated by the method of claim 1, said DDM present in a permeation-enhancing amount sufficient to substantially increase the permeability of the body surface or membrane to at least one drug in order to deliver said drug to an individual at a therapeutically effective rate.

11. The composition of claim 10, wherein the DDM and at least one drug are dispersed within a pharmaceutically acceptable carrier.

12. The composition of claim 11, wherein the DDM is combined with a permeation-enhancing amount of one or more permeation enhancers selected from monoglycerides or mixtures of monoglycerides of fatty acids, lauramide diethanolamine, lower C₁₋₄ alcohols, alkyl laurates, acyl lactylates, dodecyl acetate, and C₁₀-C₂₀fatty acid esters.

13. A composition according to claim 12 wherein the DDM is combined with a permeation-enhancing amount of one or more permeation enhancers selected from glycerol monolaurate, glycerol monooleate, glycerol monolinoleate, lauramide diethanolamine, ethanol, methyl laurate, caproyl lactylic acid, lauroyl lactylic acid, dodecyl acetate, and lauryl lactate.

14. A method comprising the steps:
a) providing a sample comprising DDM;
b) using chromatography to separate the α-anomer and the β-anomer of DDM.

15. The method of claim 14, wherein the chromatography is reverse phase HPLC.

16. The method of claim 14, wherein the chromatography is HPLC-MS or HPLC-MS-MS.

17. A method comprising the steps:
a) providing a sample comprising DDM;
b) using chromatography to detect partially deacetylated DDM.

18. The method of claim 17, wherein the chromatography is reverse phase HPLC.

19. The method of claim 17, wherein the chromatography is HPLC-MS or HPLC-MS-MS.

20. A method comprising the steps:
a) providing a sample comprising DDM;
b) using chromatography to detect dodecanol.

21. The method of claim 20, wherein the chromatography is reverse phase HPLC.

22. The method of claim 20, wherein the chromatography is HPLC-MS or HPLC-R1.

23. A method comprising the steps:
a) providing a sample comprising DDM;
b) using chromatography to detect 1-bromo or 1-chloro acylated maltose.

24. The method of claim 23, wherein the chromatography is HPLC.

25. The method of claim 24, wherein the HPLC is HPLC-MS or HPLC-MS-MS.

26. A method comprising the steps:
a) providing a sample comprising DDM;
b) using chromatography to detect acyl maltose 1-ol.

27. The method of claim 26, wherein the chromatography is HPLC.

28. The method of claim 26, wherein the chromatography is HPLC-RI, HPLC-ELSD, HPLC-Corona CAD, GC, HPLC-MS or HPLC-MS-MS.

29. A method comprising the steps:
a) providing a sample comprising DDM;
b) using chromatography to detect DDM homologues.

30. The method of claim 29, wherein the chromatography is HPLC.

31. The method of claim 29, wherein the chromatography is HPLC-RI, HPLC-ELSD, HPLC-Corona CAD, GC, HPLC-MS or HPLC-MS-MS.

32. A method comprising the steps administering to a patient a composition comprising a pharmaceutically effective amount of a drug for treating the condition and DDM, of purity as validated by the method of claim 1, in a permeation enhancing-effective amount.

33. Use of DDM, of purity as validated by the method of claim 1, as a permeation enhancing agent.
